(19) United States
(12) Patent Application Publication
(10) Pub. No.: US 2018/0298316 A1
(43) Pub. Date: Oct. 18, 2018

(54) CONTAINERS FOR CLOSED SYSTEM SINGLE-USE BIOREACTORS AND CULTURE VESSELS

(71) Applicant: ROOSTERBIO, INC., Frederick, MD (US)

(72) Inventors: Lye Theng Lock, Frederick, MD (US); Jonathan Allen Rowley, Walkersville, MD (US)

(73) Assignee: ROOSTERBIO, INC., Frederick, MD (US)

(21) Appl. No.: 15/737,906

(22) PCT Filed: Jun. 20, 2016
(86) PCT No.: PCT/US2016/038342

§ 371 Date: Dec. 19, 2017

Related U.S. Application Data
(60) Provisional application No. 62/182,183, filed on Jun. 19, 2015.

(51) Int. Cl. C12M 1/00

(52) U.S. Cl. CPC C12M 23/14; C12M 29/26

(57) ABSTRACT

A multiport container system for use in cell culture processes in the field of regenerative medicine is disclosed. Specifically, the container system can have a port that connects to a fill tubing for media and reagent addition, and additional ports for tubing and connectors which are compatible to various types of cell therapy bioreactors, single use bags, as well as different sampling and cell culture systems. The multiport container system can be used for cell production processes where a single bag could be used to fill, transport, and store media or reagents, as well as to seed and feed cells into different types of culture platform. The disclosed multiport container system can be used for the streamlining of process work flow which enable rapid process development and process transfer into cell product manufacturing.

### CROSS-REFERENCE TO RELATED APPLICATIONS

This application claims benefit of U.S. Provisional Application No. 62/182,183, filed Jun. 19, 2015, which is hereby incorporated herein by reference in its entirety.

### BACKGROUND

Disposables or single use systems are becoming the standard production platform in regenerative medicine due to the nature of therapeutic cell product that cannot be filter-sterilized. Multilayer cell factories/stacks, and single use bioreactors are the most commonly used culture platforms for therapeutic cell expansion, and is offered by various manufacturers. Often, R&D sites have multiple of these platforms being used in development or in GMP manufacturing due to various value propositions and scales that each SUB system offers. As bioproduction platform shifts towards single use systems, so is the use of media and reagents containers. Single use disposable bags are the new media and reagent storage configuration that is easy to use, and ensure the manufactured products maintain purity and sterility during storage in a scalable format. In custom designed systems, media bags can be sterile welded, sterile connected, or aseptically connected to the SUBs for media and liquid transfers that can be performed with high levels of sterility assurance.

One major challenge in the industry is the lack of standardization in tubing and aseptic connections between today's commercially available bioreactors, as they are manufactured with a wide variety of tubing and connector designs. Adding to this, media suppliers are filling media in bags that are incompatible with the tubings and connectors in single use bioreactors. This lack of standardization has led users to routinely transfer media from the media bottles or bags into bioreactor-specific media bags prior to use. This set of tasks is cumbersome, time-consuming and inefficient for users who are working with multiple culture platforms. They also present an opportunity for contamination and waste. Designing custom bags that can be filled by the media provider and are compatible with the various culture platforms that one uses can take 6-12 months, and is very costly to design, test and implement.

Alternatively, bag suppliers offer “off-the-shelf bags”, but their designs are hyper-simple and very often, cannot be filled by media manufactures or connected onto single use bioreactors. The inconsistent and non-standard bioreactor configurations in addition to their incompatibility to the media filled bag has become a major hurdle for cell therapy and other bioprocessing professionals who routinely perform testing of cell expansion in a variety of different bioreactors.

### SUMMARY

Described herein is a flexible media bag system designed with tubings and connectors that are compatible to a wide range of commercially available culture systems, and can accommodate multiple adjacent bioprocessing steps within the context of a manufacturing process. The design greatly reduces the number of processing steps typical in filling a bioreactor with cells, media, culture supplements and other reagents, greatly reducing the number of materials required for a process, lowering the cost, and greatly reducing the opportunity for a sterility breach—creating a streamlined system that solves multiple problems at once. The steps that can be performed with a single bag disclosed herein include: media filling at the media manufacturer, media storage, media supplementation, addition of cells to media, addition of microcarriers to media, seeding of media/cell/microcarriers into the bioreactor, feeding the cell cultures within the bioreactor, recovering spent media, addition of harvest reagent and harvesting the cell cultures from the bioreactor, as well as then transferring the harvested cell material into the downstream processing steps for purification, formulation, fill and freeze. This can all be accomplished with a single bag due incorporation of multiple tubings and connectors in parallel and/or in series that are compatible with a wide range of commercially available culture systems.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of an embodiment of a bag container for closed system single-use bioreactors and culture vessels.

FIG. 2 is a perspective view of an embodiment of a bag container for closed system single-use bioreactors and culture vessels.

FIG. 3 is a flow diagram of bag containers that streamlines the bioprocessing steps in cell product manufacturing.

### DETAILED DESCRIPTION

The present invention now will be described more fully hereinafter with reference to specific embodiments of the invention. The invention can be embodied in many different forms and should not be construed as limited to the embodiments set forth herein; rather, these embodiments are provided so that this disclosure will satisfy applicable legal requirements.

As used in the specification, and in the appended claims, the singular forms “a,” “an,” “the,” include plural referents unless the context clearly dictates otherwise.

The term “comprising” and variations thereof as used herein are used synonymously with the term “including” and variations thereof and are open, non-limiting terms.

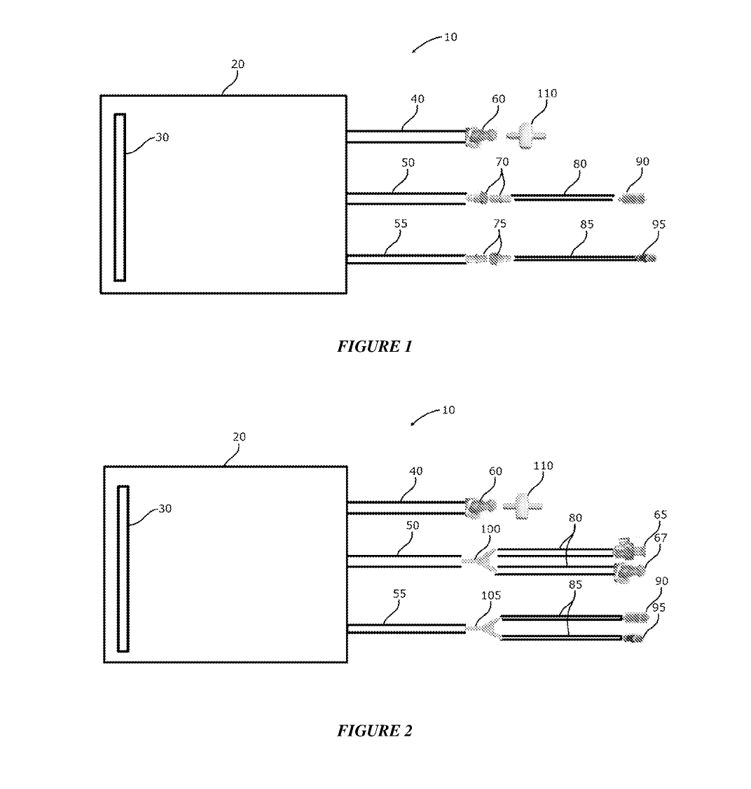

Now referring more particularly to FIGS. 1 and 2 of the drawings, a multiport-container 10 is provided that is compatible with a wide range of commercially available culture systems, and can accommodate multiple adjacent bioprocessing steps within the context of a manufacturing process.

As shown in FIGS. 1 and 2, the single use multiport-container 10 comprises a bag 20 with an interior volume configured to contain culture media. The bag 20 can therefore be made from a flexible, waterproof material, such as a plastic. In some cases, the bag 20 is made from a multilayer, laminated film having an outer layer that is puncture resistance and thermally stable. The outer layer is also preferably a radiation sterilizable material, such as polyamide, polyester or polyolefin based material. The bag 20 can also have a middle layer that is a high gas and vapor layer barrier material such as an ethylene vinyl alcohol polymer. The bag 20 can also have an inner layer for product contact which is ultra clean, pure, and low in leachables, extractables and particulates. For example, the inner layer can be made from polyethylene, ethylene vinyl acetate, or polytetrafluoroethylene (PTFE).

The bag 20 can be hermetically sealed to prevent leakage and contamination of the media. In some embodiments, the bag 20 is constructed with welded seams, in accordance with techniques that are well-known in the art, e.g., for production of plastic medical bags and the like. Other means to seal the seams of the bag 20 are also known in the art, and include, but are not limited to, heat, ultrasound and radiowave welding. Other kinds of plastic may lend themselves to other kinds of manufacture, such as mold injection.

A wide range of interior volumes is contemplated, ranging from 1 L to 500 L or more. For example, the multiport-container 10 can have a 1 L, 2 L, 5 L, 10 L, 20 L, 50 L, 100 L bag 20. However, in some case, the multiport-container 10 comprises a bag with an interior volume up to 500 L, e.g., stored in drum with optional wheels. Although a rectangular shape is illustrated in the figures, other shapes can also be used to advantage. Three-dimensional containers are also contemplated for use in the present invention. Sheets of plastic may be used to form three-dimensional containers by making more than two walls, or incorporating pleats or darts in a two-walled structure. The container may also be made of molded plastic.

The multiport-container 10 can be configured for hanging. For example, the bag 20 can be constructed with at least one hole 30 through the bag 20 for hanging the multiport-container 10. The hole 30 is sealed to prevent leakage and is optionally reinforced. In some embodiments, the bag 20 is constructed with a hook, loop, rod, magnet, fabric hook and loop fastener (e.g., Velcro®), or any other such hanging means.

The bag 20 of the disclosed multiport-container contains a plurality of tubings and connectors that are compatible with a wide range of commercially available culture systems. Examples of suitable tubing materials include C-Flex, PVC, Tygon, silicone, polyurethane or thermoplastic elastomer (TPE) tubing which can be irradiated for sterilization. The plurality of connectors can be selected based on connections being used in commercially available systems. These include, but are not limited to, quick connectors, such as MPC, CPC, or Luer lock connectors; aseptic connectors, such as AseptiQuick®, ReadyMate®, PureFit®, Opta-SFT®, KleenPak®, or Lynx S2S® connectors; or any combination thereof.

In some embodiments, the bag 20 is fluidly connected to at least one fill tubing 40 for aseptic filling of bag with culture media or reagents. The fill tubing 40 can have a proximal end fluidly connected to the bag 20 and a distal end fluidly connected to an input connector 60 for aseptic filling of bag with culture media or reagents. To maintain sterility, in some embodiments the distal end of the fill tubing or in-line filter 110 is connected to an in-line filter 110. For example, the filter can have a pore size of about 0.1-0.8 μm and surface area of about 0.0005 m²-2 m². Non-limiting examples of filter materials include polyethersulfone, PVDF, GH Polypro, polypropylene, cellulose acetate, PTFE, and nylon membrane. Once the bag 20 is filled, the fill tubing 40 can be sealed, e.g., by sterile tube welding.

The bag 20 can also be fluidly connected to at least one outlet port or dispense tubing 50, 55 for welding or connecting the bag onto culture systems such as bioreactors and multilayer cell culture systems, as well as downstream cell separation/collection systems. This can be accomplished directly, e.g., by welding, or indirectly using various combinations of tubing and connectors.

For example, the dispense tubing 50, 55 can have a proximal end fluidly connected to the bag 20 and a distal end fluidly connected to an intermediate connector 70, 75 (e.g., quick connector or Luer lock) that is configured for connecting the bag 20 onto other bag systems, sampling systems, syringes, or bioreactors. The intermediate connector 70 therefore has a proximal end fluidly connected to the dispense tubing 50, 55 and can have a distal end fluidly connected (or connectable) to connection tubing 80, 85. The connection tubing 80, 85 therefore has a proximal end fluidly connected to the intermediate connector 70, 75 and can also have a distal end that can be welded or connected onto other bag systems, sampling systems, syringes, or bioreactors. This can be accomplished, for example, using a terminal connector 90, 95 (e.g., quick connector or Luer lock). In some cases, the intermediate connector 70, 75 can be used to connect the multiport-container 10 onto different culture systems, such as bioreactors and multilayer cell culture systems, as well as downstream cell separation/collection systems, once the connection tubing 80, 85 is disconnected. Use of this intermediate connector 70, 75 provides one additional connector configuration for integration with other systems.

More than one intermediate connector 70, 75 can be used in series. Therefore, in some cases, the distal end of the connection tubing 80, 85 is fluidly connected to another intermediate connector 70 that is fluidly connected to another connection tubing 80, 85 and terminal connector 90, 95.

In some cases, the multiport-container 10 comprises at least 4, 5, 6, 7, 8, 9, 10, 11, 12 distinct configurations, sizes, or a combination thereof, of dispense tubing 50, 55, connection tubing 80, 85, intermediate connector 70, 75, and terminal connector 90, 95. For example, the multiport-container 10 can comprises at least 2, 3, 4, 5, 6, 7, 8, 9, 10, 11, 12 distinct configurations, sizes, or a combination thereof, of dispense tubing 50, 55 and connection tubing 80, 85, and can further comprise at least 2, 3, 4, 5, 6, 7, 8, 9, 10, 11, 12 distinct configurations, sizes, or a combination thereof, of intermediate connector 70, 75 and terminal connector 90, 95.

As shown in FIGS. 1 and 2, the multiport-container 10 can have at least two dispense tubing 50, 55 extending from the bag 20 in parallel. Each of these dispense tubing 50, 55 can be fluidly connected to an intermediate connector 70, 75, or directly to a terminal connector 90, 95. Moreover, each intermediate connector 70, 75 and terminal connector 90, 95 can involve different connection types and/or sizes, e.g., so that each connection is unique. For example, the multiport-container 10 in FIG. 1 has four distinctive connections 70, 75, 90, 95 for connecting to different systems. The intermediate connectors 70, 75 shown in FIG. 1 are depicted as the same type of connector but configured for different tube lumen sizes, whereas the terminal connectors 90, 95 involve different Luer lock designs.

In some cases, the multiport-container 10 can have more than two terminal connectors 65, 67, 90, 95. This can be accomplished by increasing the number of dispense tubing 50, 55 extending from the bag 20. In addition, or alternatively, as shown in FIG. 2, a splitter, such as Y-connector 100, 105, can be used to split dispense tubing 50, 55 or connection tubing 80, 85 into parallel tubings.

The above options allow the multiport-container 10 to have at least 3, 4, 5, 6, 7, 8 distinct configurations, sizes, or a combination thereof, of tubing and intermediate and terminal connectors for compatibility with different systems.

The multiport-container 10 can be pre-sterilized prior to shipping to the local user. Various sterilization techniques may be used. The choice of technique is partially dependent on the type of plastic chosen (Lee et al., 1995, in Handbook of Polymeric Biomaterials, CRC Press, Boca Raton, p 581-597). Sterilization techniques that are common in the art include, but are not limited to, dry heat, autoclaving, radiation, and ethylene oxide gas.

In preferred embodiments, the multiport-container 10 is provided to the customer with all of the tubes and connectors in place. In these embodiments, additional connectors for connecting to different systems is realized by disconnecting an intermediate connector. Contamination is less likely to be caused from removing a tube and/or connector than by adding new tubes and connectors. However, in some embodiments, the multiport-container 10 is provided to the customer with additional tubes and connectors, optionally pre-sterilized, to provide additional system configurations.

The tubings (e.g., fill tubing, dispense tubing, and connection tubing) of the disclosed multiport-container 10 can have any length suitable for bioreactors and cell culture platforms. In some cases, a tubing is not used at all. For example, the fill tubing can be replaced with a fill port or connector. Likewise, the dispense tubing can be replaced with an outlet port or connector. However, in preferred embodiments, a tubing is used with a length that allows sealing, welding, or connecting of the multiport-container 10 to various other bags, and cell culture platforms, such as cell stacks/factories, bioreactors. For example, the tubing can have a length of from 6 inches to 50 inches. The tubing also preferably has an inner diameter and wall thickness that allows welding onto identical tubings. For example, the tubing can have an inner diameter from 0.1 inch to 1 inch, and a wall thickness from 0.03 inches to 0.25 inches.

The disclosed multiport-container 10 is preferably configured for containment of media and reagents at temperature between 4-37° C. prior to use. The tubing and connectors of the disclosed multiport-container 10 can be configured, for example, for connection to a syringe or bag for process additives, connection to a cell or microcarrier inoculation system, dispensing of a solution from the interior volume of the bag into single use cell culture systems such as cell stacks/factories, and bioreactors, and solution sampling and waste or spent media collection.

For convenience and ease of use, the multiport-container 10 can be packaged and sold as a kit. Typically, a kit can contain one or more multiport-containers 10, cell culture reagents, and instructions for using the apparatus. For example, a cell expansion kit is disclosed that comprises the multiport-container disclosed herein filled with a cell culture media. The kit can further comprise a container of frozen or non-frozen cell culture supplement for adding additional nutritional and growth supplements to the media. The kit can further contain a container of frozen cells for seeing into the media bag. The kit can further contain a bag of microcarriers. The kit can further contain a bag of harvest enzyme. The kit can further contain a cell culture bioreactor or culture vessel.

In some cases, the disclosed multiport-container 10 comprises a bar code, RFID, NFC tag, or a combination, thereof for identification and tracking.

In some embodiments, the disclosed cell expansion kit is configured for generating from 50 million to 100 billion therapeutic or non-therapeutic cells per lot.

Examples of commercially available single use systems for use with the disclosed multiport-container 10 can be found in Table 1. The disclosed multiport-container is designed for compatibility with majority of these single use culture systems. Uses of the disclosed multiport-container includes, but are not limited to, media filling, containment, transportation, or storage, cells and microcarrier inoculation, cell seeding into bioreactors or multilayer vessels or feeding, cell-washing and detachment reagent as well as quench reagent and spent media containment.

TABLE 1
List of single use bioreactors or culture
vessels from different manufacturers

| MANUFACTURER | CULTURE SYSTEMS/BIOREACTORS |
|---|---|
| Corning | Cell Stacks, Cell Cube, HYPERflask |
| Thermo Scientific | Cell Factories |
| Sartorius | Univessel, BioStat STR |
| HyClone | HyPerforma SUB |
| GE Healthcare | Xuri WAVE, hollow fiber microfiltration, |
| | XCellerex |
| Pall Life Sciences | PadReactor, Xpansion, iCELLis, Nucleo SUB, |
| | Allegro STR, XRS-20 |
| PBS Biotech | Vertical-Wheel |
| EMD Millipore | Mobius CellReady |
| Eppendorf | CelligenBLU, Fibra-Cel |
| Terumo | Quantum |
| CESCO | BelloCell |
| Kuhner | Shaker |
| Cellexus | CellMaker PLUS |

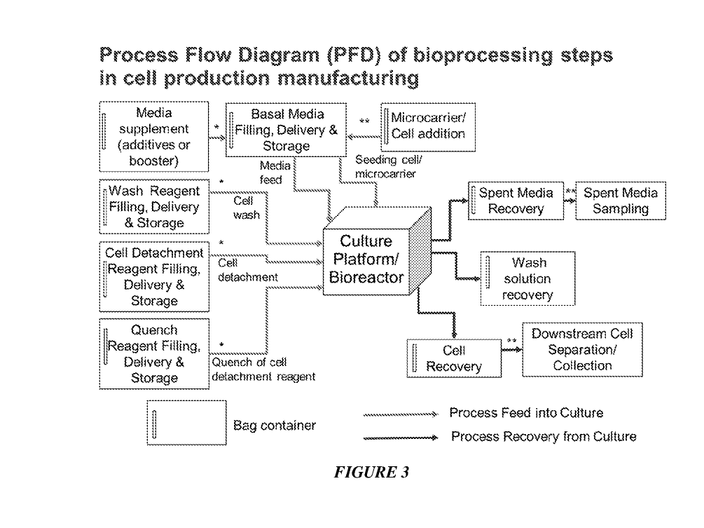

FIG. 3 is a flow diagram of bag containers which streamlines the bioprocessing steps in cell product manufacturing. The asterisk depicts where additional manipulation required in current process includes:
Transfer of media or reagents from 0.5 L bottles into bag, minimum of 3 bottles, up to 100 bottles using sterile transfer sets, which take hours to days to prepare
Uncap up to 200 media or reagent bottles and drain into larger container, and pumped into bioreactor specific bag through an adapter with filter system for re-sterilization which take hours to execute
Designing tubing adapter sets or connector sets which takes months of waiting time, and then transfer multiple media or reagents from pre-filled bag into bioreactor specific bag using the adapter sets that take hours to execute; then discarding the pre-filled bags which costs tens to hundreds of dollars each

The double asterisk depicts where additional manipulation required in current process at these marked steps includes:
Designing tubing adapter sets or connector sets which takes months of waiting time
Welding or sterilely connecting additional tubing adapter sets or connector sets that is compatible to subsequent process

A number of embodiments of the invention have been described. Nevertheless, it will be understood that various modifications may be made without departing from the spirit and scope of the invention. Accordingly, other embodiments are within the scope of the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A multiport container, comprising
a) a bag having an interior volume of from 1 L to 500 L;
b) a fill tubing having a proximal end fluidly connected to the interior volume of the bag and a distal end extending away from the bag;
c) at least one dispense tubing having a proximal end fluidly connected to the interior volume of the bag and a distal end extending away from the bag;
d) a first intermediate connector fluidly connected to the distal end of the at least one dispense tubing, and a first connection tubing having a proximal end fluidly connected to the first intermediate connector and a distal end extending away from the bag;
e) optionally a second intermediate connector fluidly connected to the distal end of the first connection tubing and a second connection tubing having a proximal end fluidly connected to the second intermediate connector and a distal end extending away from the bag; and
f) a terminal connector fluidly connected to the distal end of the first or second connection tubing;
wherein the multiport container comprises at least six distinct configurations, sizes, or a combination thereof, of dispense tubing, connection tubing, intermediate connectors, and terminal connectors.

2. The container of claim 1, wherein the intermediate connector comprises a splitter, wherein at least two first connection tubings are fluidly connected to the first intermediate connector in parallel.

3. The container of claim 1, comprising two dispense tubings extending away from the bag in parallel, each dispense tubing fluidly connected to the interior volume of the bag.

4. The container of claim 1, further comprising an in-line filter fluidly connected to the distal end of the fill tubing.

5. The container of claim 1, wherein the multiport container comprises at least six distinct configurations, sizes, or a combination thereof, of intermediate and terminal connectors.

6. The container of claim 1, wherein each of the fill tubing, dispense tubing, and connection tubing have a length of from 6 inches to 50 inches.

7. The container of claim 1, wherein each of the fill tubing, dispense tubing, and connection tubing have an inner diameter from 0.1 inch to 1 inch.

8. The container of claim 1, wherein each of the fill tubing, dispense tubing, and connection tubing have a wall thickness from 0.03 inches to 0.25 inches.

9. The container of claim 1, configured for containment of media and reagents at temperature between 4-37° C. prior to use

10. The container of claim 1, wherein the dispense tubing, extension tubing, or a combination thereof is configured for welding onto single use cell culture systems such as cell stacks/factories, bioreactors

11. The container of claim 1, wherein the intermediate connector, terminal connector, or a combination thereof, are configured for connection to a syringe or bag for process additives.

12. The container of claim 1, wherein the intermediate connector, terminal connector, or a combination thereof, are configured for connection to a cell or microcarrier inoculation system.

13. The container of claim 1, wherein the intermediate connector, terminal connector, or a combination thereof, are configured for dispensing of a solution from the interior volume of the bag into single use cell culture systems such as cell stacks/factories, and bioreactors.

14. The container of claim 1, wherein the intermediate connector, terminal connector, or a combination thereof, are configured for solution sampling and waste or spent media collection.

15. The container of claim 1, comprising a bar code, RFID, NFC tag, or a combination thereof for identification.

16. The container of claim 1, wherein the multiport container comprises at least four distinct configurations, sizes, or a combination thereof, of intermediate connectors, terminal connectors, or any combination thereof.

17. The container of claim 1, wherein the multiport container comprises at least three distinct configurations, sizes, or a combination thereof, of dispense tubing and connection tubing.

18. The container of claim 1, wherein at least one intermediate connector, terminal connector, or a combination thereof is selected from the group consisting of MPC, CPC, Luer lock, AseptiQuick®, ReadyMate®, PureFit®, Opta-SFT®, KleenPak®, Lynx S2S®.

19. A cell expansion kit comprising
a) the container of claim 1 filled with cell culture media;
b) a bag of frozen or non-frozen cell culture supplement for adding additional nutritional and growth supplements to the media;
c) optionally, a bag of frozen cells for seeding into the media bag;
d) optionally, a bag of microcarriers;
e) optionally, a bag of harvest enzyme; and
f) optionally, a cell culture bioreactor or culture vessel.

20. The cell expansion kit of claim 19, configured for generating from 50 million to 100 billion therapeutic or non-therapeutic cells per lot.
